(19) United States
(12) Patent Application Publication
(10) Pub. No.: US 2024/0393272 A1
(43) Pub. Date: Nov. 28, 2024

(54) METHODS FOR OPERATING THERMAL CONDUCTIVITY SENSORS

(71) Applicant: Infineon Technologies AG, Neubiberg (DE)

(72) Inventors: Michael KANDLER, Sauerlach (DE); Matthias EBERL, Taufkirchen (DE); Gerald HANSEKOWITSCH, St. Veit am Vogau (AT)

(21) Appl. No.: 18/665,352

(22) Filed: May 15, 2024

(30) Foreign Application Priority Data
May 25, 2023 (DE) 102023113676.3

(51) Int. Cl. G01N 25/18; G01N 33/00

(52) U.S. Cl. CPC G01N 25/18; G01N 33/0027

(57) ABSTRACT

A method for operating a thermal conductivity sensor includes the following steps: (i) applying a supply voltage to a measurement element of the thermal conductivity sensor, wherein the supply voltage results in a temperature increase of the measurement element to a characteristic temperature at which the measurement element is sensitive to a thermal conductivity of an analysis gas, (ii) performing a first measurement by the measurement element during the temperature increase before the measurement element has reached the characteristic temperature, thereby providing a first measurement value, (iii) performing a second measurement by the measurement element at a time when the measurement element has reached the characteristic temperature, thereby providing a second measurement value, and (iv) obtaining a compensated measurement value by compensating an offset of the second measurement value based on the first measurement value.

### CROSS-REFERENCE TO RELATED APPLICATION

This application claims priority to German Patent Application No. 102023113676.3 filed on May 25, 2023, the content of which is incorporated by reference herein in its entirety.

### TECHNICAL FIELD

The present disclosure relates to methods for operating thermal conductivity sensors. In addition, the present disclosure relates to thermal conductivity sensors configured to perform such methods.

### BACKGROUND

Thermal conductivity sensors may e.g., be used in the automotive sector or a variety of industrial applications. Here, the sensors may provide measurement values specifying a thermal conductivity of an analysis gas. The measurement values may suffer from offset effects that may change over the lifetime of the sensor. Manufacturers and designers of thermal conductivity sensors are constantly striving to improve their products. In particular, it may be desirable to provide thermal conductivity sensors taking into account offset effects in order to provide reliable and accurate measurement results. In addition, it may be desirable to provide suitable methods for operating such thermal conductivity sensors.

### SUMMARY

An aspect of the present disclosure relates to a method for operating a thermal conductivity sensor. The method includes the following steps: (i) applying a supply voltage to a measurement element of the thermal conductivity sensor, wherein the supply voltage results in a temperature increase of the measurement element to a characteristic temperature at which the measurement element is sensitive to a thermal conductivity of an analysis gas; (ii) performing a first measurement by the measurement element during the temperature increase before the measurement element has reached the characteristic temperature, thereby providing a first measurement value; (iii) performing a second measurement by the measurement element at a time when the measurement element has reached the characteristic temperature, thereby providing a second measurement value; and (iv) obtaining a compensated measurement value by compensating an offset of the second measurement value based on the first measurement value.

A further aspect of the present disclosure relates to a method for operating a thermal conductivity sensor. The method includes: applying a first supply voltage to a measurement element of the thermal conductivity sensor; performing a first measurement by the measurement element at the first supply voltage for an analysis gas, thereby providing a first measurement value; applying a second supply voltage to the measurement element, wherein the second supply voltage is higher than the first supply voltage; performing a second measurement by the measurement element at the second supply voltage for the analysis gas, thereby providing a second measurement value; and obtaining a compensated measurement value by compensating an offset of the second measurement value based on the first measurement value.

A further aspect of the present disclosure relates to a thermal conductivity sensor. The thermal conductivity sensor includes a measurement element configured to: perform a first measurement during a temperature increase of the measurement element to a characteristic temperature at which the measurement element is sensitive to a thermal conductivity of an analysis gas and before the measurement element has reached the characteristic temperature, thereby providing a first measurement value, perform a second measurement at a time when the measurement element has reached the characteristic temperature, thereby providing a second measurement value. The thermal conductivity sensor further includes a unit configured to obtain a compensated measurement value by compensating an offset of the second measurement value based on the first measurement value.

A further aspect of the present disclosure relates to a thermal conductivity sensor. The thermal conductivity sensor includes a measurement element configured to: perform a first measurement at a first supply voltage applied to the measurement element for an analysis gas, thereby providing a first measurement value, perform a second measurement at a second supply voltage applied to the measurement element for the analysis gas, thereby providing a second measurement value, wherein the second supply voltage is higher than the first supply voltage. The thermal conductivity sensor further includes a unit configured to obtain a compensated measurement value by compensating an offset of the second measurement value based on the first measurement value.

### BRIEF DESCRIPTION OF THE DRAWINGS

Methods and devices in accordance with the disclosure will be explained in more detail below based on the drawings. Like reference numerals designate corresponding similar parts. The features of the various illustrated examples can be combined unless they exclude each other and/or can be selectively omitted if not described to be necessarily required. Examples are depicted in the drawings and are exemplarily detailed in the description which follows.

FIGS. 1A and 1B illustrate a perspective view of a resistor 100 including a hot wire exposed to air and hydrogen, respectively.

FIG. 2 illustrates a circuit diagram of a bridge circuit 200 that may be included in a thermal conductivity resistor in accordance with the disclosure.

FIG. 3 is a diagram illustrating offset effects that may occur in a thermal conductivity sensor over its lifetime.

FIG. 4 illustrates a flowchart of a method for operating a thermal conductivity sensor in accordance with the disclosure.

FIGS. 5A-5C illustrate timing diagrams for a supply voltage, a temperature of a bridge circuit and a measurement phase during a method in accordance with the disclosure.

FIG. 6 illustrates a flowchart of a method for operating a thermal conductivity sensor in accordance with the disclosure.

FIGS. 7A-7C illustrate timing diagrams for a supply voltage, a temperature of a bridge circuit and a measurement phase during a method in accordance with the disclosure.

FIG. 8 illustrates a processing unit in accordance with the disclosure.

### DETAILED DESCRIPTION

In this description thermal conductivity sensors (or thermal conductivity gas sensors) in accordance with the disclosure and methods for operating such sensors will be described in detail. Thermal conductivity sensors as described herein may particularly be used as hydrogen sensors for detecting hydrogen and/or hydrogen concentrations. Hydrogen sensors may be used in a variety of applications, such as e.g., in the automotive sector or industrial applications. By way of example, hydrogen sensors may be used for hydrogen exhaust gas detection, exhaust gas monitoring, battery monitoring, hydrogen leakage detection, hydrogen detection in industrial plants, etc.

With a view to achieving climate targets, the automotive industry is promoting and developing the production of hydrogen-powered vehicles. Fuel cell cars can be considered as a breakthrough for electromobility and can heavily contribute to a reduced CO₂ emission. Thermal conductivity sensors as described herein improve hydrogen technology and may thus at least partially contribute to achieving climate targets that have been set. The thermal conductivity sensors as described herein provide a simple and efficient way to compensate offset effects. Compared to this, production and design of conventional sensors may be more complex and may require a higher number of components, resulting in an increased consumption of resources. The thermal conductivity sensors as described herein save resources and may contribute to energy savings. As a whole, improved thermal conductivity sensors in accordance with the disclosure and methods for operating such sensors may contribute to green technology and green power solutions, e.g., climate-friendly solutions providing reduced energy usage.

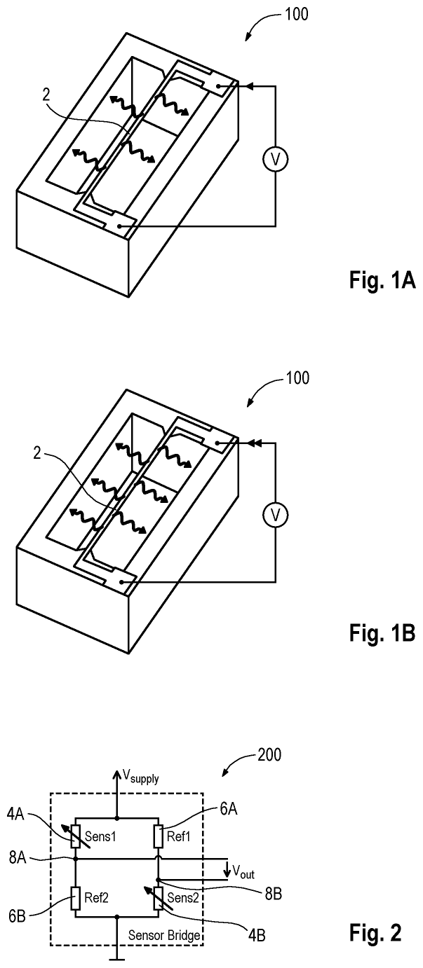

The resistor 100 of FIGS. 1A and 1B may include a hot wire (or heating wire) 2 that may be exposed to a gas. For example, the resistor 100 may be manufactured based on a MEMS (Microelectromechanical systems) technology. When a supply voltage V is applied, the hot wire 2 may heat up to a stable characteristic temperature above ambient temperature. The hot wire 2 may dissipate thermal energy to the surrounding gas as indicated by small arrows pointing away from the hot wire 2. When the stable characteristic temperature is reached, a total heat loss of the resistor 100 (or the hot wire 2) may equal the energy generated by the supply voltage V. Naturally, the heat loss of the resistor 100 may depend on the thermal conductivity of the surrounding gas. The higher the thermal conductivity of the gas, the larger a cooling effect of the surrounding gas and the lower the temperature of the resistor 100 at a constant supply voltage V. In one example, the resistor 100 may be a PTC (Positive Temperature Coefficient) resistor configured to conduct electric currents better at low temperatures than at high temperatures. That is, a resistance value of the resistor 100 may be smaller at low temperatures than at high temperatures. As a result, the electric current through the hot wire 2 may be a measure for the thermal conductivity of the surrounding gas.

FIG. 1A illustrates an operation of the resistor 100 when exposed to air (or ambient air) that may have a nitrogen content of about 78%. Thermal conductivity sensors as described herein may use nitrogen as a reference gas. A resistor exposed to a reference gas may be referred to as reference resistor. Thermal energy dissipated from the hot wire 2 into the surrounding air is indicated by small arrows.

FIG. 1B illustrates a similar operation of the resistor 100 when exposed to an analysis gas (or a gas of interest). A resistor configured to be exposed to an analysis gas may be referred to as sensor resistor. In the example of FIG. 1B, the analysis gas may be hydrogen. Note, however, that analysis gases are not restricted to a specific type and may differ in other examples. Compared to FIG. 1A, more thermal energy may be dissipated by the hot wire 2 into the surrounding hydrogen gas as indicated by a greater number of arrows. Accordingly, the electric current through the hot wire 2 in FIG. 1B may be greater than the corresponding electric current in FIG. 1A.

In many applications the electric current through the hot wire 2 may not necessarily be measured directly for analyzing a gas of interest. Instead, one or more sensor resistors exposed to an analysis gas and one or more reference resistors not exposed to the analysis gas may be combined in a half bridge circuit or a bridge circuit. Hereby, a change of thermal conductivity may be turned into a change of a bridge output voltage as discussed in connection with FIG. 2.

FIG. 2 illustrates a circuit diagram of a bridge circuit 200 that may be included in a thermal conductivity resistor in accordance with the disclosure. The bridge circuit 200 may correspond to a Wheatstone bridge circuit including two sensor resistors 4A, 4B and two reference resistors 6A, 6B. The resistors may be interconnected as shown in the circuit diagram. A resistance value of the sensor resistors 4A, 4B may change based on the presence and concentration of an analysis gas. A resistance value of the reference resistors 6A, 6B may substantially remain constant. For example, each of the resistors may be similar to the resistor 100 of FIG. 1.

The bridge circuit 200 may further include a component (not illustrated) for providing a measurement value specifying a thermal conductivity of an analysis gas. In particular, the component may be configured to measure and output a voltage difference Vₒᵤₜ between a first node 8A and a second node 8B. The first node 8A may be arranged between the first sensor resistor 4A and the second reference resistor 6B, while the second node 8B may be arranged between the second sensor resistor 4B and the first reference resistor 6A.

During an operation of the bridge circuit 200, a supply voltage may be applied as shown in FIG. 2. During a heating phase the bridge circuit 200 may heat up to a characteristic stable temperature at which the bridge circuit 200 may have become sensitive (e.g., fully sensitive) to a thermal conductivity of the analysis gas. Due to differences in the thermal conductivities of the reference gas and the analysis gas, the bridge circuit 200 may output a non-zero output voltage Vₒᵤₜ that may specify the thermal conductivity of the analysis gas. Based on the provided output voltage Vₒᵤₜ the analysis gas and/or a concentration of the analysis gas may be determined.

In the following, the bridge circuit 200 may be referred to as measurement element. It is to be noted that the bridge circuit 200 is an example and may be replaced by any other half bridge circuit or bridge circuit configured to provide a measurement value specifying the thermal conductivity of an analysis gas. Accordingly, measurement elements as described herein may correspond to or may include a bridge circuit or a half bridge circuit. Thermal conductivity sensors as described herein are not restricted to the example Wheatstone bridge circuit 200 of FIG. 2.

It is to be noted that thermal conductivity sensors as described herein may include further circuit components such as e.g., a switch, a signal amplifier, an analog digital converter, etc. However, such components may not necessarily be regarded as part of a measurement element as exemplarily shown in FIG. 2. An operation of these additional components may not necessarily depend on the present temperature. In contrast to this, an operation of the bridge circuit 200 and the value of the output voltage Vₒᵤₜ may be sensitive to the temperature of the bridge circuit.

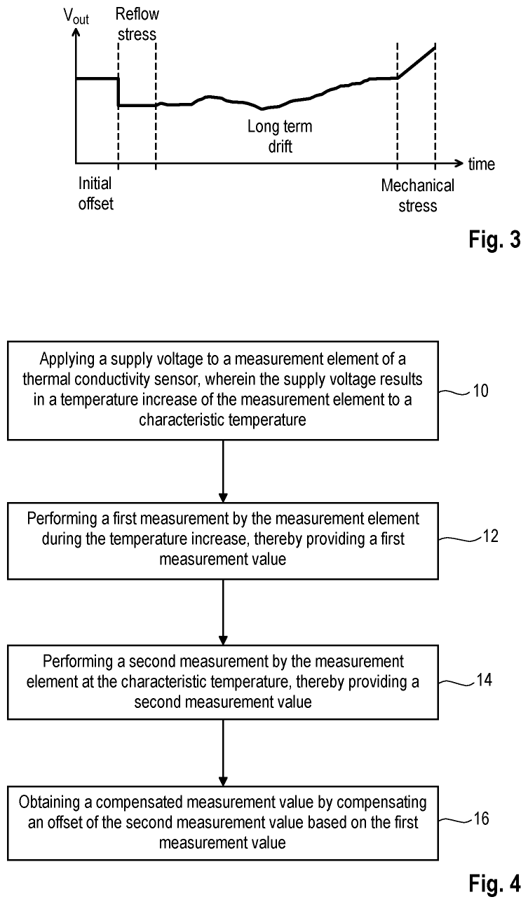

FIG. 3 is a diagram illustrating offset effects that may occur in a thermal conductivity sensor over its lifetime. An output voltage Vₒᵤₜ of the thermal conductivity sensor is plotted against time. The illustrated offset voltages may result from various effects that may occur over the lifetime of the sensor. The offset voltages may differ from sensor to sensor and may be superimposed on voltage changes induced by an analysis gas.

An initial offset voltage of the sensor may e.g., result from tolerances in the manufacturing process of the resistors included in the corresponding bridge circuit (see “Initial offset”). The manufactured sensor may be soldered (e.g., to a board), wherein an associated reflow process may result in mechanical stress impacting the resistors (see “Reflow stress”). During a comparably longer time span a long term drift of the output voltage Vₒᵤₜ may occur, which may e.g., be based on mechanical stress effects in the sensor that may change over time (see “Long term drift”). During an operation of the sensor additional mechanical stress may occur, for example based on frequently occurring temperature changes. Such stress effects are exemplarily indicated by a linear increase of the output voltage Vₒᵤₜ after the long term drift section (see “Mechanical stress”). It is to be noted that the list of offset effects discussed in connection with FIG. 3 is not necessarily complete. Further conditions may result in voltage offsets, such as e.g., pressure, humidity, etc. In the following, methods for operating thermal conductivity sensors are discussed that are capable of compensating the discussed offset effects.

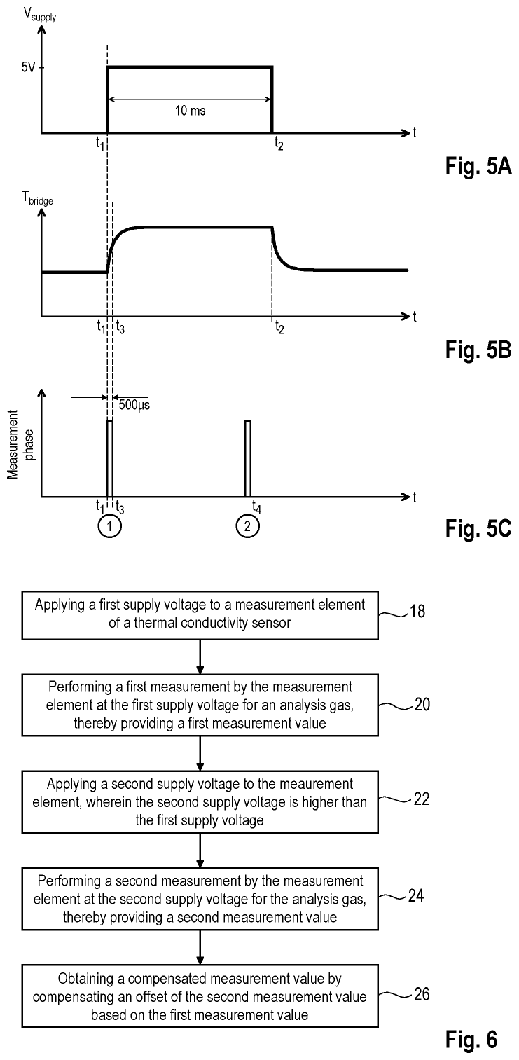

FIG. 4 illustrates a flowchart of a method for operating a thermal conductivity sensor in accordance with the disclosure. The method is described in a general manner in order to qualitatively specify aspects of the disclosure. The method may include further aspects. For example, the method may be extended by any of the aspects described in connection with the timing diagrams of FIGS. 5A to 5C.

At 10, a supply voltage may be applied to a measurement element of a thermal conductivity sensor. The supply voltage may result in a temperature increase of the measurement element to a characteristic temperature at which the measurement element is sensitive to a thermal conductivity of an analysis gas. At 12, a first measurement may be performed by the measurement element during the temperature increase before the measurement element has reached the characteristic temperature. In this connection, a first measurement value may be provided. At 14, a second measurement may be performed by the measurement element at a time when the measurement element has reached the characteristic temperature. In this connection, a second measurement value may be provided. At 16, a compensated measurement value may be obtained by compensating an offset of the second measurement value based on the first measurement value.

Thermal conductivity sensors in accordance with the disclosure may include components configured to perform the method of FIG. 4. In particular, such thermal conductivity sensors may include a measurement element configured to perform steps 12 and 14. In addition, the sensors may include a processing unit configured to obtain the compensated measurement value according to step 16. The processing unit may be configured to process two or more measurement values to generate a compensated measurement value. The processing unit may include one or more processors, an analog-to-digital converter (ADC) to convert analog measurement values to digital measurement values, a digital signal processor and/or digital processing circuitry, a logic circuit, a logic programmable gate array, and/or a field-programmable gate array (FPGA).

FIGS. 5A to 5C illustrate timing diagrams for a supply voltage, a temperature of a bridge circuit and a measurement phase during a method in accordance with the disclosure. The timing diagrams may be read in connection with the method of FIG. 4.

Referring back to step 10 of FIG. 4 and as shown in FIG. 5A, a supply voltage may be applied to a measurement element of a thermal conductivity sensor. The measurement element may e.g., correspond to the bridge circuit 200 of FIG. 2. In one example, the supply voltage may be a pulsed voltage including one or multiple voltage pulses. Amplitudes and lengths of voltage pulses as described herein may depend on various factors, such as e.g., the geometry of the considered thermal conductivity sensor, an analog to digital conversion method performed by the sensor, etc. In an increasing system as considered here, a time constant t may be specified as a time for the system's step response to reach about 1−1/e≈63.2% of its final (asymptotic) value. A system may be regarded as settled (or to be in a steady state) after a time of about five times the time constant τ, e.g., 5·τ. Accordingly, in one example, the supply voltage may include at least one voltage pulse having a minimum length of about five times a time constant τ associated with the measurement element. In the non-limiting example of FIG. 5A, the supply voltage may include a voltage pulse between times t₁ and t₂ having an amplitude of about 5V and a length of (10±20%) ms. In further examples, voltage pulses having different amplitudes and different lengths may be applied.

As can be seen from the diagram of FIG. 5B, the applied supply voltage may initiate a heating phase of the measurement element including a temperature increase of the measurement element (or the bridge circuit) to a characteristic temperature at which the measurement element may be sensitive to a thermal conductivity of an analysis gas. Before time t₁ the measurement element may be substantially at ambient temperature. At time t₁, the temperature of the measurement element may start to increase until the characteristic temperature is reached. The characteristic temperature may be a stable temperature that may have been reached when a total heat loss of the bridge circuit may equal the energy generated by the supply voltage.

Referring back to step 12 of FIG. 4 and as shown in FIG. 5C, a first measurement may be performed by the measurement element during the temperature increase at a time t₃ before the measurement element has reached the characteristic temperature. The first measurement may be carried out right after the start of the temperature increase. In particular, the first measurement may be performed (500±20%) μs after a rising edge of the voltage pulse, e.g., (500±20%) μs after time t₁. The first measurement may provide a first measurement value M₁. Referring back to FIG. 2, the provided measurement value may correspond to an output voltage of a suitable half bridge circuit or bridge circuit.

Due to the thermal inertia of the measurement element (or the thermal inertia of the bridge circuit and its resistors), the measurement element may need some time to heat up and reach the characteristic temperature. Therefore, at time t₃, the bridge may still be substantially at ambient temperature. Accordingly, the measurement element may be substantially insensitive to the thermal conductivity of the analysis gas at time t₃ when performing the first measurement. As a result, the first measurement value M₁ obtained by the first measurement may not necessarily depend on a thermal conductivity of the analysis gas. However, the first measurement value M₁ may specify (or may be based on) one or multiple offset effects as described in connection with FIG. 3.

Referring back to step 14 of FIG. 4 and as shown in FIG. 5C, a second measurement may be performed by the measurement element at a time t₄ when the measurement element has already reached the characteristic temperature. In particular, time t₄ may be before time t₂. At time t₄, the measurement element may be substantially fully sensitive to a thermal conductivity of the analysis gas. As a result, a second measurement value M₂ obtained by the second measurement may specify (or may be based on) both the thermal conductivity of the analysis gas and one or more offset effects.

Referring back to step 16 of FIG. 4, a compensated measurement value M_{comp} may be obtained based on the first measurement value M₁ and the second measurement value M₂. Since the second measurement value M₂ may take into account the thermal conductivity of the analysis gas, but the first measurement value M₁ may not, offset effects may be removed from the second measurement value M₂ by subtracting the first measurement value M₁ from it. A compensated measurement value M_{comp} may thus be obtained according to

$${M}_{comp} = {M}_{2} - {M}_{1} . \qquad ( 1 )$$

The compensated measurement value M_{comp} may be (substantially) free of one or multiple offset effects as discussed in connection with FIG. 3. In a further step, the analysis gas and/or a concentration of the analysis gas may be detected based on the compensated measurement value M_{comp}.

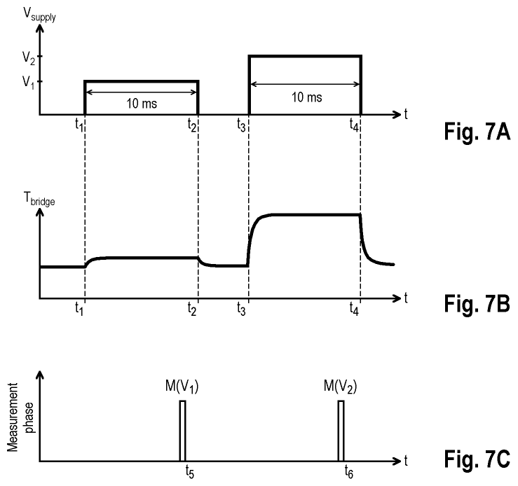

FIG. 6 illustrates a flowchart of a further method for operating a thermal conductivity sensor in accordance with the disclosure. The method is described in a general manner in order to qualitatively specify aspects of the disclosure. The method may include further aspects. For example, the method may be extended by any of the aspects described in connection with the timing diagrams of FIGS. 7A to 7C.

At 18, a first supply voltage may be applied to a measurement element of a thermal conductivity sensor. At 20, a first measurement may be performed by the measurement element at the first supply voltage for an analysis gas. In this connection, a first measurement value may be provided. At 22, a second supply voltage may be applied to the measurement element, wherein the second supply voltage is higher than the first supply voltage. At 24, a second measurement may be performed by the measurement element at the second supply voltage for the analysis gas. In this connection, a second measurement value may be provided. At 26, a compensated measurement value may be obtained by compensating an offset of the second measurement value based on the first measurement value.

Thermal conductivity sensors in accordance with the disclosure may include components configured to perform the method of FIG. 6. In particular, such thermal conductivity sensors may include a measurement element configured to perform steps 20 and 24. In addition, the sensors may include a processing unit configured to obtain the compensated measurement value according to step 26. The processing unit may be configured to process two or more measurement values to generate a compensated measurement value.

FIGS. 7A to 7C illustrate timing diagrams for a supply voltage, a temperature of a bridge circuit and a measurement phase during a method in accordance with the disclosure. The timing diagrams may be read in connection with the method of FIG. 6.

Referring back to step 18 of FIG. 6 and as shown in FIG. 7A, a first supply voltage V₁ may be applied to a measurement element of a thermal conductivity sensor. The measurement element may e.g., correspond to the bridge circuit 200 of FIG. 2. For example, the first supply voltage V₁ may be a pulsed voltage. In one example, the first supply voltage V₁ may include at least one voltage pulse having a minimum length of about five times a time constant τ associated with the measurement element. In the non-limiting example of FIG. 7A, the first supply voltage V₁ may include a voltage pulse between times t₁ and t₂ having a length of (10±20%) ms.

As can be seen from the diagram of FIG. 7B, the first supply voltage V₁ may result in a temperature increase of the measurement element to a first stable temperature. Before time t₁ the measurement element may be substantially at ambient temperature. At time t₁, the temperature of the measurement element may start to increase until the first stable temperature is reached.

Referring back to step 20 of FIG. 6 and as shown in FIG. 7C, a first measurement may be performed by the measurement element for an analysis gas at a time t₅ when the measurement element has reached the first stable temperature. In particular, time t₅ may be before time t₂. The sensitivity of the measurement element may have a cubic dependency on the applied supply voltage. Accordingly, at low supply voltages a measurement value provided by the measurement element may have almost no sensitivity to the thermal conductivity of the analysis gas. On the other hand, offset effects as described in connection with FIG. 3 may depend in an almost linear manner from the applied supply voltage. The first supply voltage V₁ may be chosen such that the measurement element may be substantially insensitive to a thermal conductivity of the analysis gas, but sensitive to offset effects, when performing the first measurement. As a result, a first measurement value M(V₁) obtained by the first measurement may not necessarily depend on the thermal conductivity of the analysis gas, but may specify (or may be based on) one or multiple offset effects as described in connection with FIG. 3.

Referring back to step 22 of FIG. 6 and as shown in FIG. 7A, a second supply voltage V₂ may be applied to the measurement element of the thermal conductivity sensor. For example, the second supply voltage V₂ may be a pulsed voltage. In one example, the second supply voltage V₂ may include at least one voltage pulse having a minimum length of about five times a time constant τ associated with the measurement element. In the non-limiting example of FIG. 7A, the second supply voltage V₂ may include a voltage pulse between times t₃ and t₄ having a length of (10±20%) ms. The second supply voltage V₂ may be higher than the first supply voltage V₁. In particular, the second supply voltage V₂ may be at least 50% higher than the first supply voltage V₁.

As can be seen from the timing diagram of FIG. 7B, the second supply voltage V₂ may result in a temperature increase of the measurement element to a characteristic second temperature at which the measurement element may be sensitive to a thermal conductivity of an analysis gas. Before time t₃ the measurement element may be substantially at ambient temperature. At time t₃, the temperature of the measurement element may start to increase until the characteristic second temperature may be reached. The characteristic second temperature may be a stable temperature that may have been reached when a total heat loss of the measurement element may equal the energy generated by the supply voltage V₂.

Referring back to step 24 of FIG. 6 and as shown in FIG. 7C, a second measurement may be performed by the measurement element at a time t₆ when the measurement element has reached the characteristic second temperature. In particular, time t₆ may be before time t₄. At time t₆, the measurement element may be substantially fully sensitive to a thermal conductivity of the analysis gas. As a result, a second measurement value M(V₂) obtained by the second measurement may specify (or may be based on) both the thermal conductivity of the analysis gas and offset effects.

Referring back to step 26 of FIG. 6, a compensated measurement value M_{comp} may be obtained by compensating an offset included in the second measurement value M(V₂) using the first measurement value M(V₁). In particular, the compensated measurement value M_{comp} may be obtained according to

$${M}_{comp} = M ( {V}_{2} ) - \frac{{V}_{2}}{{V}_{1}} \cdot M ( {V}_{1} ) \cdot {F}_{corr} . \qquad ( 2 )$$

The method discussed in connection with FIGS. 6 and 7 may be based on two different supply voltages V₁ and V₂. In equation (2), the quotient V₂/V₁ may represent a linear scaling of the measurement value with the amplitude of the associated supply voltage. In addition, an empirical correction factor F_{corr} may be used to take into consideration that mechanical stress effects, as e.g., described in connection with FIG. 3, may not scale perfectly linear with the applied supply voltage. The empirical correction factor F_{corr} may e.g., be determined from experiments. In some examples, the empirical correction factor F_{corr} may have a value of 1±10%.

Referring back to the methods of FIGS. 4 and 5, it is to be noted that these method may be performed at a single supply voltage. Therefore, the discussed linear scaling quotient V₂/V₁ and the correction factor F_{corr} need not be considered. Accordingly, equation (1) for obtaining the compensated measurement value M_{comp} may neither include a scaling quotient V₂/V₁ nor a correction factor F_{corr}.

Each of the first method of FIGS. 4 and 5 and the second method of FIGS. 6 and 7 may provide an efficient offset compensation for thermal conductivity sensors. Experiments may show that certain offset effects may be better compensated with either the first method or the second method. In some examples, it may thus be useful to provide a technique combining both concepts. As previously described, the first method of FIGS. 4 and 5 may be based on performing a measurement during the heating phase and a measurement at the stable characteristic temperature at a same supply voltage, while the second method of FIGS. 6 and 7 may be based on performing two measurements at two different voltages. A combination of the two concepts may thus be realized by performing the first method of FIGS. 4 and 5 at multiple different supply voltages.

In this connection, the method discussed in connection with FIGS. 4 and 5 may be extended in the following way. Method steps 10 to 16 may be performed for at least one further supply voltage that may differ from the supply voltage used in FIG. 5A, wherein at least one further pair of measurement values may be obtained. A compensated measurement value M_{comp} may then be obtained based on

$${M}_{comp} = {\sum }_{i = 1}^{n} {A}_{i} M ( {V}_{i} , {t}_{A} ) + {B}_{i} M ( {V}_{i} , {t}_{B} ) \qquad ( 3 )$$

In equation (3), Aᵢ and Bᵢ may be weighting factors, t_{A} and t_{B} may be the time of the first measurement and the time of the second measurement, respectively, M(Vᵢ, t_{A}) and M(Vᵢ, t_{B}) may be measurement values taken at a supply voltage Vᵢ and at times t_{A} and t_{B}, respectively, and n may be a number of supply voltages at which steps 10 to 16 of FIG. 4 may be performed. The number n may be greater than or equal to two. The weighing factors Aᵢ and Bᵢ may be positive or negative and may be determined based on a calibration of the respective thermal conductivity sensor.

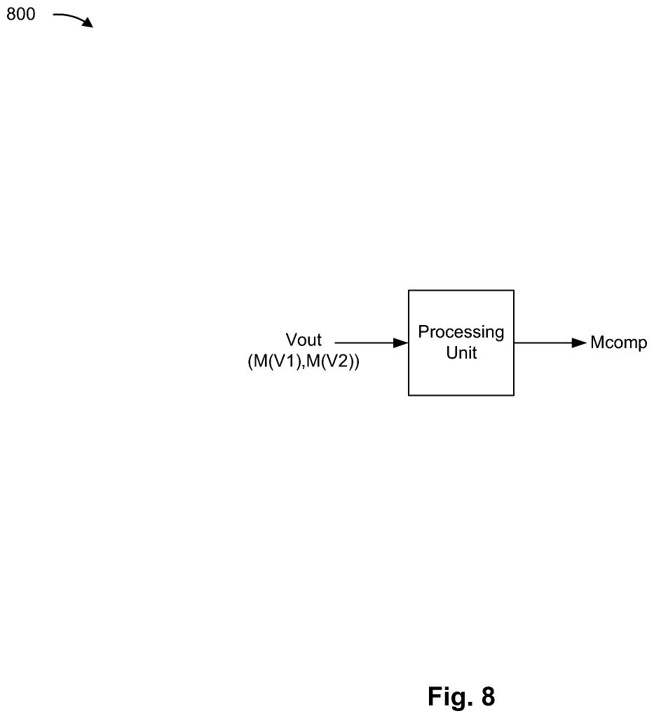

FIG. 8 illustrates a processing unit 800 in accordance with the disclosure. The processing unit 800 may receive the first and second measurement values M(V₁), M(V₂), and generate and output the compensated measurement value M_{comp} by compensating an offset of the second measurement value M(V₂) based on the first measurement value M(V₁). Thus, the processing unit 800 may be coupled to the bridge circuit 200 (e.g., the measurement element) for receiving the output voltage Vₒᵤₜ. The processing unit 800 may obtain the first measurement value M(V₁) by a first measurement of the bridge circuit 200 (e.g., a first measurement of the output voltage Vₒᵤₜ), and may obtain the second measurement value M(V₁) by a second measurement of the bridge circuit 200 (e.g., a second measurement of the output voltage Vₒᵤₜ), as disclosed herein. The processing unit 800 may be configured to process the first measurement value M(V₁) and the second measurement value M(V₂) to generate the compensated measurement value M_{comp}, as disclosed herein. The processing unit 800 may include one or more processors, an analog-to-digital converter (ADC) to convert analog measurement values to digital measurement values, a digital signal processor and/or digital processing circuitry, a logic circuit, a logic programmable gate array, and/or a field-programmable gate array (FPGA).

### Aspects

In the following, methods for operating thermal conductivity sensors and thermal conductivity sensors in accordance with the disclosure will be explained using aspects.

Aspect 1 is a method for operating a thermal conductivity sensor, the method comprising the following steps: (i) applying a supply voltage to a measurement element of the thermal conductivity sensor, wherein the supply voltage results in a temperature increase of the measurement element to a characteristic temperature at which the measurement element is sensitive to a thermal conductivity of an analysis gas; (ii) performing a first measurement by the measurement element during the temperature increase before the measurement element has reached the characteristic temperature, thereby providing a first measurement value; (iii) performing a second measurement by the measurement element at a time when the measurement element has reached the characteristic temperature, thereby providing a second measurement value; and (iv) obtaining a compensated measurement value by compensating an offset of the second measurement value based on the first measurement value.

Aspect 2 is a method of Aspect 1, wherein compensating the offset of the second measurement value comprises: subtracting the first measurement value from the second measurement value.

Aspect 3 is a method of Aspect 1 or 2, wherein the measurement element is substantially insensitive to the thermal conductivity of the analysis gas when performing the first measurement.

Aspect 4 is a method of one of the preceding Aspects, wherein the measurement element is substantially at ambient temperature when performing the first measurement.

Aspect 5 is a method of one of the preceding Aspects, wherein the offset is based on at least one of reflow stress, long term drift, mechanical stress, pressure, humidity.

Aspect 6 is a method of one of the preceding Aspects, wherein the measurement element comprises a bridge circuit or a half bridge circuit.

Aspect 7 is a method of Aspect 6, wherein the first measurement value and the second measurement value are output voltages of the bridge circuit or the half bridge circuit.

Aspect 8 is a method of one of the preceding Aspects, wherein: the supply voltage comprises a voltage pulse having a minimum length of about five times a time constant associated with the measurement element, and the first measurement is performed (500±20%) μs after a rising edge of the voltage pulse.

Aspect 9 is a method of one of the preceding Aspects, further comprising: performing steps (i) to (iv) for at least one different supply voltage, thereby obtaining at least one further pair of measurement values, wherein obtaining the compensated measurement value is further based on the at least one further pair of measurement values.

Aspect 10 is a method of Aspect 9, wherein the compensated measurement value is obtained based on M_{comp}=Σᵢ₌₁ⁿ AᵢM(Vᵢ, t_{A})+BᵢM(Vᵢ, t_{B}), wherein: M_{comp} is the compensated measurement value, Aᵢ and Bᵢ are weighting factors, t_{A} and t_{B} are the time of the first measurement and the time of the second measurement, respectively, M(Vᵢ, t_{A}) and M(Vᵢ, t_{B}) are measurement values taken at a supply voltage Vᵢ and at times t_{A} and t_{B}, respectively, and n is a number of supply voltages at which steps (i) to (iv) are performed.

Aspect 11 is a method of Aspect 10, further comprising: determining the weighing factors Aᵢ and Bᵢ based on calibrating the thermal conductivity sensor.

Aspect 12 is a method of one of the preceding Aspects, further comprising: detecting the analysis gas and/or a concentration of the analysis gas based on the compensated measurement value.

Aspect 13 is a method for operating a thermal conductivity sensor, the method comprising: applying a first supply voltage to a measurement element of the thermal conductivity sensor; performing a first measurement by the measurement element at the first supply voltage for an analysis gas, thereby providing a first measurement value; applying a second supply voltage to the measurement element, wherein the second supply voltage is higher than the first supply voltage; performing a second measurement by the measurement element at the second supply voltage for the analysis gas, thereby providing a second measurement value; and obtaining a compensated measurement value by compensating an offset of the second measurement value based on the first measurement value.

Aspect 14 is a method of Aspect 13, wherein the second supply voltage is at least 50% higher than the first supply voltage.

Aspect 15 is a method of Aspect 13 or 14, wherein: the measurement element is substantially insensitive to a thermal conductivity of the analysis gas when performing the first measurement, and the measurement element is substantially fully sensitive to the thermal conductivity of the analysis gas when performing the second measurement.

Aspect 16 is a method of one of Aspects 13 to 15, wherein the compensated measurement value is obtained based on

$${M}_{comp} = M ( {V}_{2} ) - \frac{{V}_{2}}{{V}_{1}} \cdot M ( {V}_{1} ) \cdot {F}_{corr} ,$$

wherein: V₁ and V₂ are the first supply voltage and the second supply voltage, respectively, M(V₁) and M(V₂) are the first measurement value and the second measurement value, respectively, and F_{corr} is an empirical correction factor.

Aspect 17 is a method of Aspect 16, wherein the empirical correction factor has a value of 1±10%.

Aspect 18 is a thermal conductivity sensor, comprising: a measurement element configured to: perform a first measurement during a temperature increase of the measurement element to a characteristic temperature at which the measurement element is sensitive to a thermal conductivity of an analysis gas and before the measurement element has reached the characteristic temperature, thereby providing a first measurement value, perform a second measurement at a time when the measurement element has reached the characteristic temperature, thereby providing a second measurement value; and a processing unit configured to obtain a compensated measurement value by compensating an offset of the second measurement value based on the first measurement value.

Aspect 19 is a thermal conductivity sensor of Aspect 18, wherein the measurement element comprises a Wheatstone bridge.

Aspect 20 is a thermal conductivity sensor of Aspect 19, wherein the Wheatstone bridge comprises four resistors and each of the resistors comprises a hot wire.

Aspect 21 is a thermal conductivity sensor, comprising: a measurement element configured to: perform a first measurement at a first supply voltage applied to the measurement element for an analysis gas, thereby providing a first measurement value, perform a second measurement at a second supply voltage applied to the measurement element for the analysis gas, thereby providing a second measurement value, wherein the second supply voltage is higher than the first supply voltage; and a processing unit configured to obtain a compensated measurement value by compensating an offset of the second measurement value based on the first measurement value.

While this implementation has been described with reference to illustrative aspects, this description is not intended to be construed in a limiting sense. Various modifications and combinations of the illustrative aspects, as well as other aspects of the implementation, will be apparent to persons skilled in the art upon reference to the description. It is therefore intended that the appended claims encompass any such modifications or aspects.

1. A method for operating a thermal conductivity sensor, the method comprising the following steps:
(i) applying a supply voltage to a measurement element of the thermal conductivity sensor, wherein the supply voltage results in a temperature increase of the measurement element to a characteristic temperature at which the measurement element is sensitive to a thermal conductivity of an analysis gas;
(ii) performing a first measurement by the measurement element during the temperature increase before the measurement element has reached the characteristic temperature, thereby providing a first measurement value;
(iii) performing a second measurement by the measurement element at a time when the measurement element has reached the characteristic temperature, thereby providing a second measurement value; and
(iv) obtaining a compensated measurement value by compensating an offset of the second measurement value based on the first measurement value.

2. The method of claim 1, wherein compensating the offset of the second measurement value comprises:
subtracting the first measurement value from the second measurement value.

3. The method of claim 1, wherein the measurement element is substantially insensitive to the thermal conductivity of the analysis gas when performing the first measurement.

4. The method of claim 1, wherein the measurement element is substantially at ambient temperature when performing the first measurement.

5. The method of claim 1, wherein the offset is based on at least one of reflow stress, long term drift, mechanical stress, pressure, humidity.

6. The method of claim 1, wherein the measurement element comprises a bridge circuit or a half bridge circuit.

7. The method of claim 6, wherein the first measurement value and the second measurement value are output voltages of the bridge circuit or the half bridge circuit.

8. The method of claim 1, wherein:
the supply voltage comprises a voltage pulse having a minimum length of about five times a time constant associated with the measurement element, and
the first measurement is performed 500±20% μs after a rising edge of the voltage pulse.

9. The method of claim 1, further comprising:
performing steps (i) to (iv) for at least one different supply voltage, thereby obtaining at least one further pair of measurement values, wherein obtaining the compensated measurement value is further based on the at least one further pair of measurement values.

10. The method of claim 9, wherein the compensated measurement value is obtained based on:
$${M}_{comp} = {\sum }_{i = 1}^{n} {A}_{i} M ( {V}_{i} , {t}_{A} ) + {B}_{i} M ( {V}_{i} , {t}_{B} ) .$$
wherein:
M_{comp} is the compensated measurement value,
Aᵢ and Bᵢ are weighting factors,
t_{A} and t_{B} are the time of the first measurement and the time of the second measurement, respectively,
M(Vᵢ, t_{A}) and M(Vᵢ, t_{B}) are measurement values taken at a supply voltage Vᵢ and at times t_{A} and t_{B}, respectively, and
n is a number of supply voltages at which steps (i) to (iv) are performed.

11. The method of claim 10, further comprising:
determining the weighing factors Aᵢ and Bᵢ based on calibrating the thermal conductivity sensor.

12. The method of claim 1, further comprising:
detecting the analysis gas and/or a concentration of the analysis gas based on the compensated measurement value.

13. A method for operating a thermal conductivity sensor, the method comprising:
applying a first supply voltage to a measurement element of the thermal conductivity sensor;
performing a first measurement by the measurement element at the first supply voltage for an analysis gas, thereby providing a first measurement value;
applying a second supply voltage to the measurement element, wherein the second supply voltage is higher than the first supply voltage;
performing a second measurement by the measurement element at the second supply voltage for the analysis gas, thereby providing a second measurement value; and
obtaining a compensated measurement value by compensating an offset of the second measurement value based on the first measurement value.

14. The method of claim 13, wherein the second supply voltage is at least 50% higher than the first supply voltage.

15. The method of claim 13, wherein:
the measurement element is substantially insensitive to a thermal conductivity of the analysis gas when performing the first measurement, and
the measurement element is substantially fully sensitive to the thermal conductivity of the analysis gas when performing the second measurement.

16. The method of claim 13, wherein the compensated measurement value is obtained based on
$${M}_{comp} = M ( {V}_{2} ) - \frac{{V}_{2}}{{V}_{1}} \cdot M ( {V}_{1} ) \cdot {F}_{corr}$$
wherein:
V₁ and V₂ are the first supply voltage and the second supply voltage, respectively,
M(V₁) and M(V₂) are the first measurement value and the second measurement value, respectively, and
F_{corr} is an empirical correction factor.

17. The method of claim 16, wherein the empirical correction factor has a value of 1±10%.

18. Thermal conductivity sensor, comprising:
a measurement element configured to:
perform a first measurement during a temperature increase of the measurement element to a characteristic temperature at which the measurement element is sensitive to a thermal conductivity of an analysis gas and before the measurement element has reached the characteristic temperature, thereby providing a first measurement value,
perform a second measurement at a time when the measurement element has reached the characteristic temperature, thereby providing a second measurement value; and
a processing unit configured to obtain a compensated measurement value by compensating an offset of the second measurement value based on the first measurement value.

19. The thermal conductivity sensor of claim 18, wherein the measurement element comprises a Wheatstone bridge.

20. The thermal conductivity sensor of claim 19, wherein the Wheatstone bridge comprises four resistors and each of the four resistors comprises a hot wire.

21. A thermal conductivity sensor, comprising:
a measurement element configured to:
perform a first measurement at a first supply voltage applied to the measurement element for an analysis gas, thereby providing a first measurement value,
perform a second measurement at a second supply voltage applied to the measurement element for the analysis gas, thereby providing a second measurement value, wherein the second supply voltage is higher than the first supply voltage; and
a processing unit configured to obtain a compensated measurement value by compensating an offset of the second measurement value based on the first measurement value.
